(19) United States
(12) Patent Application Publication
(10) Pub. No.: US 2004/0175775 A1
(43) Pub. Date: Sep. 9, 2004

(54) Method of detecting PrPsc in eye fluid

(76) Inventor: Neil Cashman, Toronto (CA)

Correspondence Address: CLARK & ELBING LLP, 101 FEDERAL STREET, BOSTON, MA, 02110, US

(21) Appl. No.: 10/704,228

(22) Filed: Nov. 6, 2003

Related U.S. Application Data
(60) Provisional application No. 60/424,983, filed on Nov. 8, 2002.

(51) Int. Cl. G01N 33/53; G01N 33/537; G01N 33/543

(52) U.S. Cl. 435/7.92

(57) ABSTRACT

The invention provides novel methods of detecting PrP^{Sc}. More specifically, the invention provides novel methods of detecting PrP^{Sc} in eye fluid extracted from an eye globe of an animal. In contrast to previously known methods, the methods of the invention are readily performed on living animals, thereby facilitating the diagnosis of PrP^{Sc}-associated disease prior to death. The methods also feature the decreased presence of antigen-binding factors, which could block antibody-antigen interactions, as well as lower background PrP^{c}.

### CROSS REFERENCES TO RELATED APPLICATIONS

This application claims benefit of U.S. provisional patent application No. 60/424,983, filed on Nov. 8, 2002.

### BACKGROUND OF THE INVENTION

1. Field of the Invention

The invention relates to the fields of medicine, veterinary medicine, genetics, biochemistry and molecular biology. In particular, the invention relates to methods of detecting PrP^{Sc}. More specifically, the invention relates to methods of detecting PrP^{Sc} in eye fluid extracted from an eye globe of an animal.

2. Description of the Related Art

Prions are infectious pathogens that cause central nervous system spongiform encephalopathies in humans and animals, including Creutzfeldt-Jakob Disease (CJD), bovine spongiform encephalopathy (BSE, or “mad cow disease”), and scrapie. Prions are distinct from bacteria, viruses and viroids. The prevailing hypothesis at present is that prion infectivity is due to a template directed protein conformational change, and not dependent upon a nucleic acid component.

A major advance in the study of prions and the diseases that they cause was the discovery and purification of a protein designated prion protein (“PrP”). Complete prion protein-encoding genes have since been cloned, sequenced and expressed in transgenic animals. The normal isoform of PrP is called PrP^{C} (the cellular form of PrP). PrP^{C} is encoded by a single-copy host gene and is normally found at the outer surface of cells, most abundantly in neurons. The transmissible spongiform encephalopathies (TSE) of humans and animals are accompanied by the conversion of PrP^{C} into a modified form generically called PrP^{Sc} (the scrapie-specific isoform of PrP), and the accumulation of PrP^{Sc} in nervous tissues and other organs. The actual biological or physiological function of PrP^{C} is not known.

The scrapie isoform of the prion protein (PrP^{Sc}) is necessary for both the transmission and pathogenesis of the TSEs of animals and humans. The most common prion diseases of animals are scrapie in sheep and goats and BSE in cattle. Five prion diseases of humans have been identified: kuru, CJD, Gerstmann-Strassler-Scheinker Disease (GSS), fatal familial insomnia (FFI), and variant CJD (vCJD). The presentation of human prion diseases as sporadic, genetic and infectious illnesses initially posed a conundrum, which has been explained by the “protein only” hypothesis of prion infectivity.

The importance of animal prion diseases is illustrated by BSE or “mad cow disease” in Great Britain, where more than 150,000 cattle have died and serious consideration has been given to slaughtering millions of cattle potentially infected with prions. BSE is thought to have originated with cattle consuming meat and bone meal produced from sheep offal that contained scrapie prions. In the late 1970s, the hydrocarbon-solvent extraction method used in the rendering of offal began to be abandoned, resulting in meat and bone meal with a much higher fat content. It is now thought that this change in the rendering process allowed scrapie prions from sheep to survive rendering and to be transmitted to cattle. See U.S. Pat. No. 6,008,435.

In humans, most prion diseases occur sporadically, but about 10-15% are inherited as autosomal dominant disorders that are caused by mutations in the human PrP gene (PRNP). Additionally, iatrogenic CJD has been caused by human growth hormone (hGH) derived from cadaveric pituitaries as well as dura matter grafts. That the hGH prepared from pituitaries was contaminated with prions is supported by the transmission of prion disease to a monkey 66 months after inoculation with a suspect lot of hGH. The long incubation times associated with prion diseases will not reveal the full extent of iatrogenic CJD for decades in thousands of people treated with hGH worldwide. See U.S. Pat. No. 6,372,214 B1.

It now seems possible that bovine prions from “mad cows” was passed to humans through the consumption of tainted beef products, leading to CJD in some individuals. Although many plans have been offered for the culling of older cattle in order to minimize the spread of BSE, it seems more important to monitor the frequency of prion disease in cattle as they are slaughtered for human consumption. Immunoblotting of the brainstems of cattle for PrP^{Sc} provides an approach to establish the incidence of subclinical BSE in cattle entering the human food chain.

At least two groups have disclosed methods of detecting PrP^{Sc} in live animals. O'Rourke et al. describes an immunological assay for the preclinical detection of PrP^{Sc} in biopsies of nictitating membrane (third eyelid) lymphoid tissue of sheep. See U.S. Pat. No. 6,261,790; see also O'Rourke et al. (1999) The Veterinary Record, May 2, 1998, pages 489-491. In addition, U.S. Pat. No. 6,150,172, issued to Schmerr et al., describes the extraction of prion protein from several biological fluids, including cerebrospinal fluid, blood, serum, plasma, milk, urine, saliva, tears, mucous secretions, sweat, and semen. However, neither O'Rourke et al. nor Schmerr et al. teaches methods for detecting PrP^{Sc} in eye fluid. Detection methods useful in live animals could prevent the costly wholesale slaughter of herds whose BSE status is unknown to eliminate infected livestock, as has been performed in the past. Furthermore, live detection methods, even if used on stunned animal prior to killing, could prevent cross-contamination of samples or spread of infectivity, since current tests are based on brain tissue, which must be obtained on the killing floor from decapitated animals whose skulls have been opened.

At least some of these detection methods, however, could be hindered by the presence of antibody-antigen blocking factors in the chosen biological fluid, which can inhibit binding of PrP^{Sc} (see, for example, Fischer et al. (2000) Nature 408: 479-83), and may require processing to remove background PrP^{c}. Both are problems that could affect the accuracy, reproducibility, and dependability of such a detection method.

Thus, there is a need for new methods of detecting PrP^{Sc} in animals. Specifically, there is a need for convenient, cost-effective methods of detecting PrP^{Sc} in the easily accessible biological fluids of animals, particularly if those fluids are free of antibody-antigen blocking factors and contain minimal PrP^{c}. The present invention provides such methods.

### BRIEF SUMMARY OF THE INVENTION

The invention relates to methods of detecting PrP^{Sc}. More specifically, the invention relates to methods of detecting PrP^{Sc} in eye fluid extracted from an eye globe of an animal.

In one embodiment, the invention provides an improved method of detecting PrP^{Sc} in an animal, the improvement comprising detecting PrP^{Sc} in eye fluid extracted from an eye globe of an animal.

In a preferred embodiment, the invention comprises: extracting eye fluid from an eye globe of an animal; contacting the eye fluid with an antibody capable of binding to PrP^{Sc}; forming detectable PrP^{Sc}-antibody complexes; and detecting PrP^{Sc}-antibody complexes; wherein detection of PrP^{Sc}-antibody complexes indicates the presence of PrP^{Sc} in the eye fluid of the animal.

In a further preferred embodiment, the invention comprises a step wherein prior to forming PrP^{Sc}-antibody complexes, the eye fluid is first contacted with a protease capable of distinguishing PrP^{Sc} from PrP^{C}. For example, proteinase K is such a protease.

### BRIEF DESCRIPTION OF THE DRAWINGS

FIG. 1 is an autoradiogram showing the results of immunoprecipitation of hamster scrapie PrP^{Sc} from human eye fluid. Lanes are designated 1-12 from left to right. The migration of molecular weight protein size markers (kDa) is indicated at the right side of the figure.

### DETAILED DESCRIPTION OF THE INVENTION

This invention comprises methods of detecting PrP^{Sc} in the body fluids of animals. In a preferred embodiment, this invention comprises the detection of PrP^{Sc} in accessible biological fluids, including eye fluid contained in the eyeball (eye globe) of humans and animals. PrP^{Sc} may be detected in retina of cattle with bovine spongiform encephalopathy (Bradley R, Dev Biol Stand 1999; 99: 35-40). PrP^{Sc} shed from the retina may also detectable in eye fluids (vitreous and aqueous humor) that are in direct or diffusion contact with the retina. The eye globes of humans and animals are readily accessible to devices designed to obtain eye fluid samples, and prions may be present in a higher concentration in eye fluid than in blood or urine.

The methods of the invention may be used to detect PrP^{Sc} in any body fluid that contains PrP^{Sc}, including eye fluid. Extraction of eye fluid from the eye globe of an animal may be by any method known to those of skill in the art. Exemplary eye fluids are the aqueous humor and the vitreous humor. Moreover, the methods of the invention may be used to detect PrP^{Sc} in or from any animal. In addition to humans, examples of animals in which PrP^{Sc} may be detected using the methods of the invention include all mammals such as a cow, sheep, horse, pig, dog, chicken, and cat.

Numerous methods of detecting PrP^{Sc} are known to those of skill in the art, any of which may be used in the methods of this invention. For example, PrP^{Sc} may be detected by virtue of partial protease resistance, although not all species of PrP^{Sc} possess this property. PrP^{Sc} differs from PrP^{C} in its protease resistance: upon treatment with an appropriate protease, such as proteinase K, PrP^{C} is destroyed while PrP^{Sc} is reduced from its original size of 32-35 kDa to a smaller size of 27-30 kDa. The remaining protease-resistant PrP^{Sc} fragment is referred to as PrP 27-30. See, for example, International Publication No. WO 99/42829.

Additionally, PrP^{Sc} may be detected by affinity reagents, such as antibodies (see, for example, International Publication No. WO 00/78344), other prion binding proteins (see, for example, International Publication No. WO 97/45746), and peptides (see, for example, U.S. Utility application No. 10/256,538). For example, International Publication No. WO 98/37210 discloses monoclonal antibodies, including antibody 6H4, useful in immunological assays for the identification of prions; U.S. Pat. No. 5,846,533 discloses antibodies that specifically bind to native PrP^{Sc} in situ and in vitro; and International Publication No. WO 93/11155 discloses synthetic polypeptides having at least one antigenic site of a prion protein and antibodies raised against such polypeptides.

As used herein, “antibody” refers to an immunoglobulin protein or modification thereof whether naturally, recombinantly, or synthetically produced, which is capable of specifically binding an antigen. In the course of natural production, antibodies are secreted into the bloodstream to seek out and bind foreign agents or antigens for destruction. The term also covers any protein having a binding domain that is homologous to an immunoglobulin binding domain, such as an antibody fragment (a portion of an antibody (i.e. Fv) capable of binding to an antigen), recombinant CDR domain, or a cyclized peptide corresponding to a CDR domain.

As used herein, “protein”, “peptide, or “polypeptide” refers to a number of naturally occurring, or synthetically or recombinantly produced, some times extremely complex (such as an enzyme or antibody) substances that consist of a chain of four or more amino acid residues joined by peptide bonds. The chain may be linear, branched, circular, or combinations thereof. Intra-protein bonds also include disulfide bonds. Protein molecules contain the elements carbon, hydrogen, nitrogen, oxygen, usually sulfur, and occasionally other elements (such as phosphorus or iron). Herein, “protein” (and its given equivalent terms) is also considered to encompass fragments, variants and modifications (including, but not limited to, glycosylated, acylated, myristylated, and/or phosphorylated residues) thereof, including the use of amino acid analogs, as well as non-proteinacious compounds intrinsic to enzymatic function, such as co-factors, or guide templates (for example, the template RNA associated with proper telomerase function).

“Specific binding”, or when something is said to “specifically bind” and/or “specifically interact” refers to binding, even briefly, between a protein, and one or more other proteins, molecules, and/or compounds, wherein the interaction is dependent upon the primary amino acid sequence, conformation, or modifications to the protein. It may also refer to binding to self, or other molecules of the same protein, as in the forming of dimers and other multimers.

As used herein, “prion binding protein” refers to full-length proteinacious gene products that are not antibodies or antibody fragments, and to peptides or protein fragments greater than 200 amino acids in length. Descriptions of such proteins may be found in International Publication No. WO 97/45746. “Prion binding peptide” refers to the peptides of the invention disclosed in U.S. patent application Ser. No. 10/256,538. Such peptides are generally 200 or fewer amino acids in length.

Contacting eye fluid, or preparations, extractions, precipitates, or fractions thereof, with PrP^{Sc} binding antibodies, other prion binding proteins, and/or peptides capable of binding to PrP^{Sc} may be under any appropriate conditions to permit, or that can be subsequently be changed or altered to permit, complex formation between PrP^{Sc} and the binding molecule(s). By “binding” is meant a covalent and/or non-covalent interaction (such as hydrogen bonding, ionic interactions among charged groups or dipoles, van der Waals interactions, and hydrophobic interactions among non-polar groups) that holds, even briefly, two or more molecules together. Eye fluid or components thereof may be presented with a solid support or carrier. Exemplary solid supports useful in the methods of the invention include magnetic beads or other beads (e.g., agarose), membranes (e.g. nitrocellulose, nylon), or plate wells (e.g., ELISA, or derivatized surfaces), carriers would generally be soluble substrates, such as proteins (such as albumin or thyroglobulin) or other soluble molecules. Under conditions where PrP^{C} is present, binding of PrP^{Sc} is preferably selective binding, wherein the binding molecule preferentially binds to PrP^{Sc}, as compared to binding PrP^{c}, for a given set of conditions. Complexes formed should be detectable complexes, or precursors to detectable complexes.

Detection of PrP^{Sc}-antibody complexes, PrP^{SC}-PrP^{SC} binding protein complexes and/or PrP^{SC}-PrP^{SC} binding peptide complexes, may be by any methods known in the art. For example, detection methods may make use of any appropriate label which may be directly or indirectly visualized may be utilized in these detection assays including, without limitation, any epitope tag, radioactive, fluorescent, chromogenic (e.g., alkaline phosphatase or horseradish peroxidase), or chemiluminescent label, or a hapten (for example, digoxigenin or biotin) which may be visualized using a labeled, hapten-specific antibody or other binding partner (e.g., avidin). Or, for further example, methods which may rely on direct or indirect changes in a sample's optical characteristics or migratory characteristics when examined by chromatography and the like, such as light scattering methodologies, tryptophan fluorescence, UV absorption, turbidity measurements, a filter retardation assay, size exclusion chromatography, reversed-phase high performance liquid chromatography, a fluorescent binding assay, a protein-staining assay, microscopy, or polyacrylamide gel electrophoresis (PAGE).

In one example, the PRIONICS-CHECK® test kit (Prionics Product No. 12000; Roche Cat. No. 3184986) can be used. This kit combines the above strategies and monitors three independent criteria: protease-resistance, glycosylation pattern and lower molecular weight of the protease-resistant PrP^{Sc} fragment (27-30 kDa) compared to normal, undigested PrP. In a first step, protease treatment converts PrP^{Sc} to the PrP 27-30 fragment while destroying PrP^{C}. In a second step, PrP 27-30 is identified by its immunoreactivity with anti-PrP antibodies and by its size, in an optimized Western blotting procedure.

Further examples of the above methods of detecting PrP^{Sc} are described in the following articles: Schaller, O. et al. (1999) Acta Neuropathologica 98: 437-443; Veterinary Record 145: 672; Oesch, B. et al. (2000) Archives of Virology Supp. 16: 189-195.

The following examples are presented for illustrative purposes only and are not intended, nor should they be construed, as limiting the invention in any way. Those skilled in the art will recognize that variations on the following can be made without exceeding the spirit or scope of the invention.

### EXAMPLE 1

### Immunoprecipitation of Hamster Scrapie PrP^{Sc} from Human Eye Fluid

Human eye fluid obtained during ophthalmologic procedures was mixed with small quantities of brain tissue taken from a scrapie-infected hamster, and magnetic bead-conjugated antibodies were used to immunoprecipitate this material (see International Publication No. WO 00/78344 for immunoprecipitation protocol). Immunoprecipitated product was eluted from beads and subjected to immunoblotting analysis using the PrP-reactive antibody 3F4.

More specifically, three different sample mixtures were first produced: (a) 10 μL phosphate-buffered saline (PBS) and 90 μL human eye fluid; (b) 10 μL 10% hamster scrapie brain homogenate and 90 μL human eye fluid; and (c) 10 μL 10% hamster scrapie brain homogenate and 90 μL PBS. Next, 50 μL antibody-bound magnetic beads and 850 μL binding buffer (6% detergent) was added to each of the three 100 μL mixtures. Four different types of antibody-bound magnetic beads were used in the immunoprecipitation, each with a different antibody bound to the beads: 6H4 (see Korth et al. Nature 1997 Nov. 6; 390(6655): 74-7), which is PrP isoform non-selective and therefore binds to both PrP^{Sc} and PrP^{C}; 16A 18 (see International Publication No. WO 00/78344), which selectively binds PrP^{Sc}; 17D4 (see, International Publication No. WO 00/78344), which likewise selectively binds PrP^{Sc}; and 4E4 (see International Publication No. WO 00/78344), which is an isotype control antibody that does not bind to PrP. Following the immunoprecipitation step, the magnetic beads were isolated from the mixture and protein-antibody complexes were eluted from the magnetic beads. The antibody-bound proteins were then separated according to size by SDS-polyacrylamide gel electrophoresis. Finally, the PrP-reactive antibody 3F4 (see Kascsak et al. J Virol 1987 December; 61(12): 3688-93) was used in conventional immunoblotting analysis to visualize the immunoprecipitated proteins.

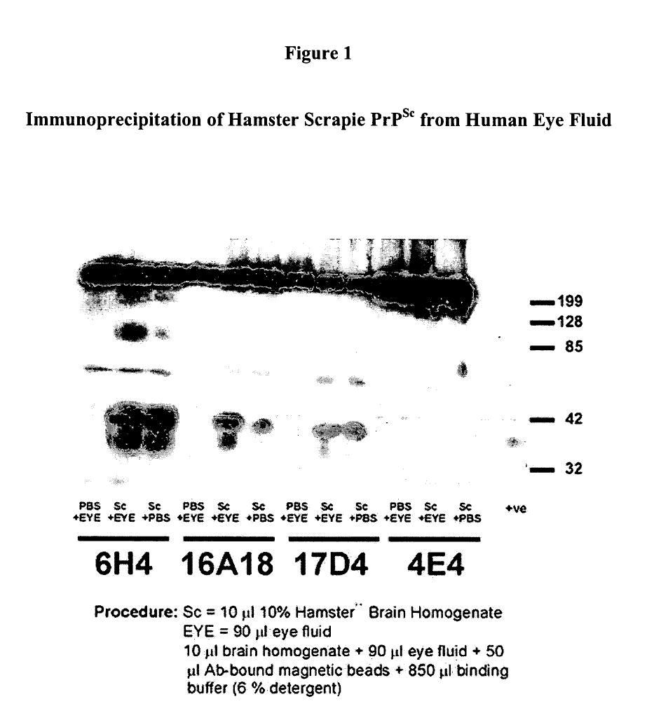

As shown in FIG. 1, the PrP isoform non-selective mAb 6H4 precipitated hamster PrP^{Sc} from eye fluid (lane 2) and PBS (lane 3), each mixed with hamster scrapie brain homogenate, in similar yields, but little or no PrP^{C} from fluids mixed with PBS (lane 1). The PrP^{Sc} selective antibodies 16A18 and 17D4 immunoprecipitated PrP^{Sc} from eye fluid (lanes 5 and 8) and PBS (lanes 6 and 9), each mixed with hamster scrapie brain homogenate, in similar yields, but precipitated no PrP from eye fluid mixed with PBS(lanes 4 and 7). The isotype control antibody 4E4 precipitated no PrP from spiked or unspiked eye fluid or PBS, each mixed with hamster scrapie brain homogenate (lanes 10-12).

These data indicate that human eye fluid contains little or no PrP^{C}, the normal isoform of the prion protein, at the level of sensitivity of the experiment. These data also indicate that PrP^{Sc} is immunoprecipitable from eye fluid without sample pretreatment, indicating the paucity of antibody-antigen blocking factors, which can inhibit binding of PrP^{Sc} specific antibodies in blood and blood fractions.

All of the articles, books, patents, patent applications, and other references cited in this patent application are hereby incorporated by reference.

Although certain presently preferred embodiments of the invention have been described herein, it will be apparent to those of skill in the art to which the invention pertains that variations and modifications of the described embodiment may be made without departing from the spirit and scope of the invention. Accordingly, it is intended that the invention be limited only to the extent required by the following claims and the applicable rules of law.

What is claimed is:

1. A method of detecting PrP^{Sc} or a fragment thereof in an animal, the method comprising the step of detecting PrP^{Sc} in eye fluid from an eye globe of the animal.

2. The method of claim 1, comprising:
(a) extracting eye fluid from an eye globe of an animal;
(b) contacting the eye fluid with an antibody capable of binding to PrP^{Sc};
(c) forming detectable PrP^{Sc}-antibody complexes; and
(d) detecting PrP^{Sc}-antibody complexes;
wherein detection of PrP^{Sc}-antibody complexes indicates the presence of PrP^{Sc} in the eye fluid of the animal.

3. The method of claim 2, wherein the PrP^{Sc} binding antibody selectively binds PrP^{Sc} as compared to PrP^{C}.

4. The method of claim 2, wherein prior to forming PrP^{Sc}-antibody complexes, the eye fluid is first contacted with a protease capable of distinguishing PrP^{Sc} from PrP^{C}.

5. The method of claim 4, wherein the protease is proteinase K.

6. The method of claim 1, comprising:
(a) extracting eye fluid from an eye globe of an animal;
(b) contacting the eye fluid with a protein capable of binding to PrP^{Sc};
(c) forming detectable PrP^{Sc}-PrP^{Sc} binding protein complexes; and
(d) detecting PrP^{Sc}-PrP^{Sc} binding protein complexes;
wherein detection of PrP^{Sc}-PrP^{Sc} binding protein complexes indicates the presence of PrP^{Sc} in the eye fluid of the animal.

7. The method of claim 6, wherein the PrP^{Sc} binding protein selectively binds PrP^{Sc} as compared to PrP^{C}.

8. The method of claim 6, wherein the PrP^{Sc} binding protein is protocadherin-43 (PC2) or a fragment thereof.

9. The method of claim 6, wherein prior to forming PrP^{Sc}-antibody complexes, the eye fluid is first contacted with a protease capable of distinguishing PrP^{Sc} from PrP^{C}.

10. The method of claim 9, wherein the protease is proteinase K.

11. The method of claim 1, comprising:
(a) extracting eye fluid from an eye globe of an animal;
(b) contacting the eye fluid with a peptide of 200 or fewer amino acids capable of binding to PrP^{Sc};
(c) forming detectable PrP^{Sc}-PrP^{Sc} binding peptide complexes; and
(d) detecting PrP^{Sc}-PrP^{Sc} binding peptide complexes;
wherein detection of PrP^{Sc}-PrP^{Sc} binding peptide complexes indicates the presence of PrP^{Sc} in the eye fluid of the animal.

12. The method of claim 11, wherein the PrP^{Sc} binding peptide selectively binds PrP^{Sc} as compared to PrP^{C}.

13. The method of claim 11, wherein said peptide is about 9 to 25 amino acids in length.

14. The method of claim 11, wherein said peptide includes a YYX (where X is any amino acid) or YSA motif.

15. The method of claim 14, wherein said YYX (where X is any amino acid) or YSA motif is repeated in said peptide.

16. The method of claim 15, wherein said YYX (where X is any amino acid) or YSA motif is tandemly repeated in said peptide.

17. The method of claim 14, wherein said YYX (where X is any amino acid) motif is selected from the group consisting of YYR, YYD, YYA, and YYQ.

18. The method of claim 11, wherein prior to forming PrP^{Sc}-PrP^{Sc} binding peptide complexes, the eye fluid is first contacted with a protease capable of distinguishing PrP^{Sc} from PrP^{C}.

19. The method of claim 18, wherein the protease is proteinase K.

20. The method of claim 1, wherein the eye fluid consists essentially of aqueous humor.

21. The method of claim 1, wherein the eye fluid consists essentially of vitreous humor.

22. The method of claim 1, wherein the animal is a mammal selected from the group consisting of a human, a cow, a sheep, a horse, a pig, a dog, a chicken, and a cat.

23. The method of claim 22, wherein the animal is a cow.

24. A method for diagnosing a prion disease in a mammal, the method comprising the step of testing eye fluid of said mammal for the presence of PrP^{Sc}, whereby detection of PrP^{Sc} in the eye fluid is indicative of the presence a prion disease in the mammal.
